(19) United States
(12) Patent Application Publication
(10) Pub. No.: US 2003/0232396 A1
(43) Pub. Date: Dec. 18, 2003

(54) Method and use of protein microarray technology and proteomic analysis to determine efficacy of human and xenographic cell, tissue and organ transplant

(75) Inventors: Aby J. Mathew, Binghamton, NY (US); John M. Baust, Candor, NY (US); Robert VanBuskirk, Apalachian, NY (US); John G. Baust, Candor, NY (US)

Correspondence Address: ANTHONY MIELE, PALMER & DODGE, LLP, 111 HUNTINGTON AVENUE, BOSTON, MA, 02199, US

(73) Assignee: BioLife Solutions, Inc.

(21) Appl. No.: 10/372,579

(22) Filed: Feb. 21, 2003

Related U.S. Application Data
(60) Provisional application No. 60/358,386, filed on Feb. 22, 2002.

(51) Int. Cl. G01N 33/53; G01N 33/567; C12M 1/34

(52) U.S. Cl. 435/7.2; 435/287.2

(57) ABSTRACT

The present invention is directed to systems and methods for assessing the success of the transplant of a cell, tissue, or organ before and after transplant. Protein array technology is used to obtain a biomarker pattern for the cell, tissue, or organ that is being considered for transplant or that has been transplanted. Samples for the identification of biomarkers and biomarker patterns are obtained from the cell, tissue or organ itself, or from a body fluid of the donor or recipient. Sample biomarker data are compared to reference biomarker data obtained from donors, recipients or cells, tissues or organs that have been transplanted. Correlation of a sample biomarker pattern with the reference biomarker pattern, where transplant outcome for the samples used for the reference biomarkers is known, permits a suggested treatment determination. A computerized system to identify the condition of transplant before or after implantation is also provided.

### FIELD OF THE INVENTION

Aspects of the present invention relate to tools and methods to assess success of a cell, tissue, or organ transplant.

### DISCUSSION OF BACKGROUND INFORMATION

There are many types of evaluations and tests used in the cell, tissue, and organ transplantation process. Pre-operative tests focus on the overall health of the transplant recipient. These tests may include blood tests for tissue typing and to determine that the patient is free of infection or other conditions that would contraindicate transplantation (e.g., cancer) as well as, for example, electrocardiograms and echocardiograms to evaluate cardiac status and tests to evaluate the patient's immune status. Ultrasound images may also be taken to check for overall health, or for the condition of areas of the body relating to the transplant site. For example, a kidney transplant recipient may undergo abdominal and renal ultrasounds to check the abdominal area, the gall bladder, and the kidneys.

Post-operative testing focuses on the success or rejection of the cells, tissues, or organs that were involved in the transplant. Blood tests are done to evaluate the function of the transplant and the health of the transplant recipient. Biopsies of the transplant may be taken to evaluate the health and function of the new cells, tissues, or organs. If the patient's body is found to be rejecting the transplant, medical intervention is called for in the form of anti-rejection drug therapies.

Protein microarray technology is being used in a number of ways to study proteins, including protein-protein interactions, protein reactions with drugs, and the quantity of various proteins in a sample. Determining the quantity of proteins in a sample is achieved through the use of arrays of capture agents that bind with the proteins in the sample. Analysis of the amount and location of the bound proteins on the array can be used in a variety of proteomic research approaches.

Von Eggeling, et al. (2000, BioTechniques 29: 1066-1070) reported the utilization of ProteinChip™ (Ciphergen, Fremont, Calif.) microarray technology for the analysis of cancerous tissue protein profiles. That study described the use of protein microarray analysis for distinguishing between cancerous and normal tissue. Other reports on the utilization of protein microarray technology for the identification of candidate genes involved in tissue repair/regeneration, disease diagnosis, as well as cancer biomarker identification further support the role of high-through put protein analysis in research and clinical settings (Li e al., 2000, Biochim. Biophys. Acta 1524: 102-109; Tonge et al., 2001, Proteomics 1: 377-396; Vlahou et al., 2001, Am. J. Pathol. 158: 1491-1502).

Hampel, et al. (2001, J. Am. Soc. Nephrol. 12: 1026-1035), reported on the utilization of ProteinChip™ microarray technology for the screening of urine as a diagnostic tool to assess renal dysfunction following administration of radiocontrast medium for cardiac function imaging.

### SUMMARY OF THE INVENTION

The present invention is directed to systems and methods for assessing the success of the transplant of a cell, tissue, or organ and provides a means to determine the health of the cell, tissue, or organ to be transplanted, and the health of the cell, tissue, or organ after it has been transplanted. The health of the patient who has received the transplanted cell, tissue, or organ can also be determined. A mechanism is also in place to make a treatment determination.

In one aspect of the invention, protein array technology is used to obtain a biomarker pattern for the cell, tissue, or organ that is being used in the transplant. A sample is placed on a platform that holds a capture agent. The proteins in the sample will bind to certain capture agents on the platform, and using a detection mechanism, the amount of each of the relevant proteins in the sample can be quantified to generate a biomarker pattern. This biomarker pattern is compared to a reference pattern or to a protein difference map, which is created, for example, by comparing the biomarker patterns of a healthy transplant to a rejected transplant. The comparison comprises a measurement of the presence, absence, or amount of the plurality of biomarkers in the two samples. The comparison of the biomarker pattern from the transplant sample and the reference pattern or the protein difference map gives information about the health of the cell, tissue, or organ involved in the transplant. This information is used to determine the course of treatment during the transplantation and recovery.

In one aspect, an apparatus is provided for assessing success of a transplant of a cell, tissue, or organ, the apparatus comprising: a holder to hold at least one of a surface chemistry and a capture agent necessary to detect a plurality of different polypeptides of a sample; a detection mechanism to determine polypeptide detection data comprising at least one of quantity and type of polypeptides bound to the holder; and a processor comprising a comparison mechanism to compare the polypeptide detection data from the sample with a reference and a mechanism to determine a condition of the cell, tissue, or organ to be transplanted based on the comparison of the polypeptide detection data from the sample with the reference.

In one embodiment, the holder comprises one of a planar surface, a bead or a cylinder.

In another embodiment, the holder comprises a microarray.

In another embodiment, the surface chemistry or capture agent comprises an antibody.

In another embodiment, the surface chemistry comprises an ion exchange or reversed-phase affinity agent.

In another embodiment, the sample comprises a sample from a cell, tissue, or organ to be transplanted.

In another embodiment, the detection mechanism comprises SELDI-TOF.

In another embodiment, the detection mechanism comprises a labeled antibody.

In another embodiment, the detection mechanism comprises surface plasmon resonance.

In another aspect, an apparatus is provided for assessing success of a transplant of a cell, tissue, or organ, the apparatus comprising: a holder to hold at least one of a surface chemistry and a capture agent necessary to detect a plurality of different polypeptides of a sample; a detection mechanism to determine polypeptide detection data comprising at least one of quantity and type of polypeptides bound to the holder; and a processor comprising a comparison mechanism to compare the polypeptide detection data from the sample with a reference, a mechanism to determine a condition of the cell, tissue, or organ that has been transplanted and a mechanism for making a treatment determination.

In one embodiment, the holder comprises one of a planar surface, a bead or a cylinder.

In another embodiment, the sample comprises a sample from the cell, tissue, or organ that has been transplanted.

In another embodiment, the sample comprises a fluid sample from the patient who has received the cell, tissue, or organ.

In another aspect, a method of evaluating the medical condition of a cell, tissue, or organ to be used as a transplant is provided, comprising: providing a tissue matched cell, tissue or organ to be transplanted; using a polypeptide array to measure the amount of a plurality of polypeptides in a sample from the cell, tissue or organ, thereby determining a pattern; and comparing the pattern of the plurality of polypeptides from the cell, tissue, or organ to the values for a reference pattern of the plurality of polypeptides; wherein a difference between the pattern observed for said transplant and the reference pattern is indicative of the medical condition of the transplant.

In another aspect, a method of evaluating the medical condition of a cell, tissue, or organ to be used as a transplant is provided, comprising: providing a cell, tissue, or organ to be used for transplant; performing matching to assess transplant donor to recipient; comparing a plurality of biomarkers from a cell, tissue, or organ to be used in a transplant to the values for a reference pattern of the plurality of biomarkers; wherein a difference between the pattern observed for said transplant and the reference pattern is indicative of the medical condition of the transplant.

In one embodiment, the comparing step comprises measurement of the presence, absence, or amount of the plurality of biomarkers.

In another embodiment, the plurality of biomarkers is at least four.

In another embodiment, the measurement is performed using a protein array.

In another embodiment, the protein array is a microarray.

In another embodiment, the microarray comprises a plurality of antibodies.

In another embodiment, the array comprises an ion exchange or reversed-phase affinity agent.

In another embodiment, the cell, tissue, or organ is a kidney, and the plurality of biomarkers comprises one or more of albumin, IgA, IgGm urokinase, thyroxine binding globulin, transferrin, anti-thrombin 3, protein S, protein C, amylase, chlecalcitol, Bence Jones protein, ribonuclease, and hemoglobin.

In another embodiment, the cell, tissue, or organ is a liver, and the plurality of biomarkers comprises one or more of aspartate aminotransferase, alanine aminotransferase, bilirubin, glutamate dehydrogenase, malate dehydrogenase, ketose-1-phosphate aldolase, and lactate dehydrogenase.

In another embodiment, the cell, tissue, or organ is a heart, and the plurality of biomarkers comprises one or more of creatine kinase, aspartate amino transferase, lactic acid dehydrogenase, and fructose aldolase.

In another embodiment, the cell, tissue, or organ is a pancreas or pancreatic islet cell, and the plurality of biomarkers comprises one or more of amylase, lipase, aspartame aminotransferase, alanine aminotransferase, lactic acid dehydrogenase, alkaline phosphatase, leucine amidopeptidase, insulin, proinsulin, and glucose phosphate isomerase.

In another aspect, a method of generating a protein difference map is provided, comprising: identifying a first biomarker pattern from a first cell, tissue, or organ; identifying a second biomarker pattern from a second cell tissue or organ, wherein the first and second cell, tissue or organ are at different stages of transplantation; and comparing said first and second biomarker patterns, thereby generating a protein difference map.

In one embodiment, the steps of identifying a biomarker pattern each comprise measuring the presence, absence, or amount of a plurality of biomarkers in a sample.

In another embodiment, the first and second biomarker patterns comprise information regarding at least four biomarkers.

In another embodiment, the biomarker pattern is identified using a microarray.

In another embodiment, the first and second cell, tissue, or organ are each the same type of cell, tissue, or organ.

In another embodiment, the first biomarker pattern is derived from a healthy transplant and the second biomarker pattern is derived from a rejected transplant.

In another embodiment, the cell, tissue, or organ is a kidney, and the biomarker pattern comprises information regarding one or more of albumin, IgA, IgGm urokinase, thyroxine binding globulin, transferrin, anti-thrombin 3, protein S, protein C, amylase, chlecalcitol, Bence Jones protein, ribonuclease, and hemoglobin.

In another embodiment, the cell, tissue, or organ is a liver, and the biomarker pattern comprises information regarding one or more of aspartate aminotransferase, alanine aminotransferase, bilirubin, glutamate dehydrogenase, malate dehydrogenase, ketose-1-phosphate aldolase, and lactate dehydrogenase.

In another embodiment, the cell, tissue, or organ is a heart, and the biomarker pattern comprises information regarding one or more of creatine kinase, aspartate amino transferase, lactic acid dehydrogenase, and fructose aldolase.

In another embodiment, the cell, tissue, or organ is a pancreas or pancreatic islet cell, and the biomarker pattern comprises information regarding one or more of amylase, lipase, aspartame aminotransferase, alanine aminotransferase, lactic acid dehydrogenase, alkaline phosphatase, leucine amidopeptidase, insulin, proinsulin, and glucose phosphate isomerase.

In another aspect, a method of predicting the suitability of a cell, tissue, or organ for transplant is provided, the method comprising: measuring the presence, absence, or amount of a plurality of polypeptide biomarkers in a cell, tissue, or organ being evaluated for transplant, to generate a biomarker pattern; and comparing said biomarker pattern to a protein difference map representing the differences in presence, absence, or amount of said plurality of biomarkers exhibited in healthy versus unhealthy cells, tissues, or organs of the same kind, wherein said comparing predicts the suitability of said cell, tissue, or organ.

In one embodiment, measuring is performed using a microarray.

In another embodiment, the microarray comprises a plurality of antibodies.

In another embodiment, the plurality of biomarkers is at least four.

In another embodiment, the cell, tissue, or organ is a kidney, and the plurality of biomarkers comprises one or more of albumin, IgA, IgGm urokinase, thyroxine binding globulin, transferrin, anti-thrombin 3, protein S, protein C, amylase, chlecalcitol, Bence Jones protein, ribonuclease, and hemoglobin.

In another embodiment, the cell, tissue, or organ is a liver, and the plurality of biomarkers comprises one or more of aspartate aminotransferase, alanine aminotransferase, bilirubin, glutamate dehydrogenase, malate dehydrogenase, ketose-1-phosphate aldolase, and lactate dehydrogenase.

In another embodiment, the cell, tissue, or organ is a heart, and the plurality of biomarkers comprises one or more of creatine kinase, aspartate amino transferase, lactic acid dehydrogenase, and fructose aldolase.

In another embodiment, the cell, tissue, or organ is a pancreas or pancreatic islet cell, and the plurality of biomarkers comprises one or more of amylase, lipase, aspartame aminotransferase, alanine aminotransferase, lactic acid dehydrogenase, alkaline phosphatase, leucine amidopeptidase, insulin, proinsulin, and glucose phosphate isomerase.

In another aspect, a protein difference map for evaluating materials for transplant, made as disclosed herein, is provided.

In another aspect, a computerized system is provided to identify a condition of a cell, tissue or organ to be transplanted, the system comprising: a stored representation of biomarker data to be assessed; a stored representation of reference biomarker data; a user interface; the user interface comprising a biomarker information input mechanism to allow a user to specify information regarding the biomarker data to be assessed; the user interface further comprising a comparison process option input mechanism to allow a user to specify a set of comparison process options; a mechanism to compare the biomarker data to be assessed with the reference biomarker data in accordance with the specified comparison process options; and a mechanism to indicate a likelihood of a successful transplant of a cell, tissue or organ to be transplanted based on the comparison of the biomarker data to be assessed and the reference biomarker data.

In one embodiment, the options comprise designation of a specific subset of the biomarker data to be assessed, and designation of the source of the sample from which the reference data were obtained. In another embodiment, the source is one of a transplant recipient, a transplant donor, a cell to be transplanted, a tissue to be transplanted, an organ to be transplanted, a transplanted cell, a transplanted tissue, and a transplanted organ. In another embodiment, the source of the sample is urine, serum, plasma or saliva from a transplant donor or transplant recipient, or storage fluid for a cell, tissue or organ to be transplanted.

In another embodiment, the mechanism to indicate a likelihood of a successful transplant displays a graphical representation of comparison results on a computer screen.

In another embodiment, the mechanism to indicate a likelihood of a successful transplant further provides a suggested transplant approach, the approach comprising a suggestion to proceed with the transplant, a suggestion to proceed with the transplant with heightened monitoring, or a suggestion not to proceed with the transplant.

In another aspect, computerized system is provided to identify a condition of a transplanted cell, tissue or organ, the system comprising: a stored representation of biomarker data to be assessed; a stored representation of reference biomarker data; a user interface; the user interface comprising a biomarker information input mechanism to allow a user to specify information regarding the biomarker data to be assessed; the user interface further comprising a comparison process option input mechanism to allow a user to specify a set of comparison process options; a mechanism to compare the biomarker data to be assessed with the reference biomarker data in accordance with the specified comparison process options; and a mechanism to indicate a condition of a transplanted cell, tissue or organ based on the comparison of the biomarker data to be assessed and the reference biomarker data.

In another embodiment, the options comprise designation of a specific subset of the biomarker data to be assessed, and designation of the source of the sample from which the reference data were obtained. In another embodiment, the source is one of a transplant recipient, a transplant donor, a cell to be transplanted, a tissue to be transplanted, an organ to be transplanted, a transplanted cell, a transplanted tissue, and a transplanted organ. In another embodiment, the source of the sample is urine, serum, plasma or saliva from a transplant donor or transplant recipient, or storage fluid for a cell, tissue or organ.

In another embodiment, the mechanism to indicate a condition displays a graphical representation of comparison results on a computer screen.

In another embodiment, the mechanism to indicate a condition further provides a suggested treatment approach, the approach comprising a suggestion to proceed with standard monitoring, a suggestion to consider initiation of aggressive drug intervention, or a suggestion to initiate aggressive drug intervention.

### BRIEF DESCRIPTION OF THE DRAWINGS

FIG. 1 shows a schematic diagram of an apparatus for assessing the status of a cell, tissue or organ before or after transplant.

FIG. 2 shows a schematic diagram of one example of a method of generating a protein difference map.

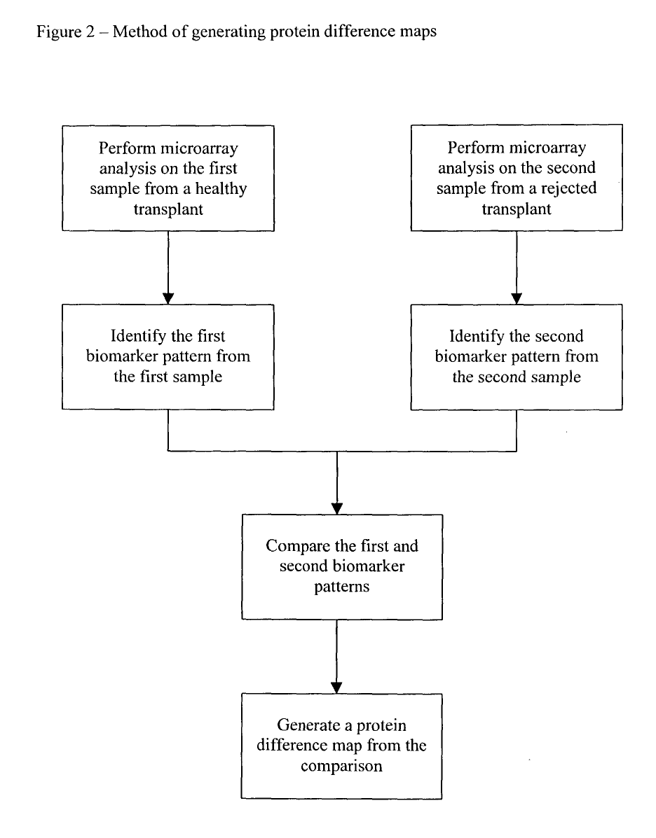

FIG. 3 shows protein spectra of purified Insulin and Glucagon protein standards analyzed on Normal Phase 1 (NP1) protein chip arrays. Standard analysis was performed as a means of assessing the accuracy of the ProteinChip™ system in comparison with reported molecular weight values. In addition, Insulin standards (20 fmol) were analyzed to determine detection variation within and between array spots on the NP1 chips. Glucagon standards were spotted in varying concentrations (6 and 20 fmol) to determine the sample detection sensitivity of the protein chips.

FIG. 4 shows protein spectra obtained from analysis of preservation medium at various time points during preservation. Analysis of fresh and transport preservation medium (Spectra A and B, respectively) revealed a relative flat line spectra pattern indicating minimal protein presence. Analysis of preservation medium flushed from kidneys revealed the presence of a substantial amount of protein present in the solution, which continued to increase, as well as the development of new protein peaks as the preservation interval extended.

FIG. 5 shows protein spectra of urinary cellular lysate samples obtained from renal transplant donor and recipient patients prior to (donor) and following (recipient) successful transplantation. Donor analysis yielded a base line profile for comparative purposes. Analysis of recipient patient samples revealed an increase in the profile intensity correlating to an increase in protein expression 24 hours and the appearance of unique proteins 48 hours after transplantation. Continued analysis at 72 hours revealed a marked decrease in protein levels which represented a return to levels similar to that of the initial donor profile.

FIG. 6 shows a schematic diagram of a process performed by a computerized system for identifying the condition of a cell, tissue or organ that is transplanted or is being considered for transplant. A set of stored biomarker data for the cell, tissue or organ to be assessed, or a specific subset of stored biomarker data is chosen. This can include the set-up of a detection process to provide the desired set of data and/or an overinclusive set of data. Once assessment is started, the chosen biomarker data to be assessed are accessed, corresponding reference data are accessed, the biomarker data to be assessed is compared to the reference data, and an indication of the condition of the cell, tissue or organ is graphically displayed, based on the comparison. The system can also make a suggestion regarding transplant or post-transplant treatment approach, including a suggestion to proceed or not proceed with the transplant, a suggestion to proceed with the transplant with heightened monitoring for one or more indicators of potential problems, a suggestion to consider initiation of aggressive drug intervention for the transplanted material, or a suggestion to initiate aggressive drug intervention for the transplanted material. The suggestions are based on the comparison of biomarker data to be assessed and reference biomarker data in light of the known outcome of treatment for the reference biomarkers.

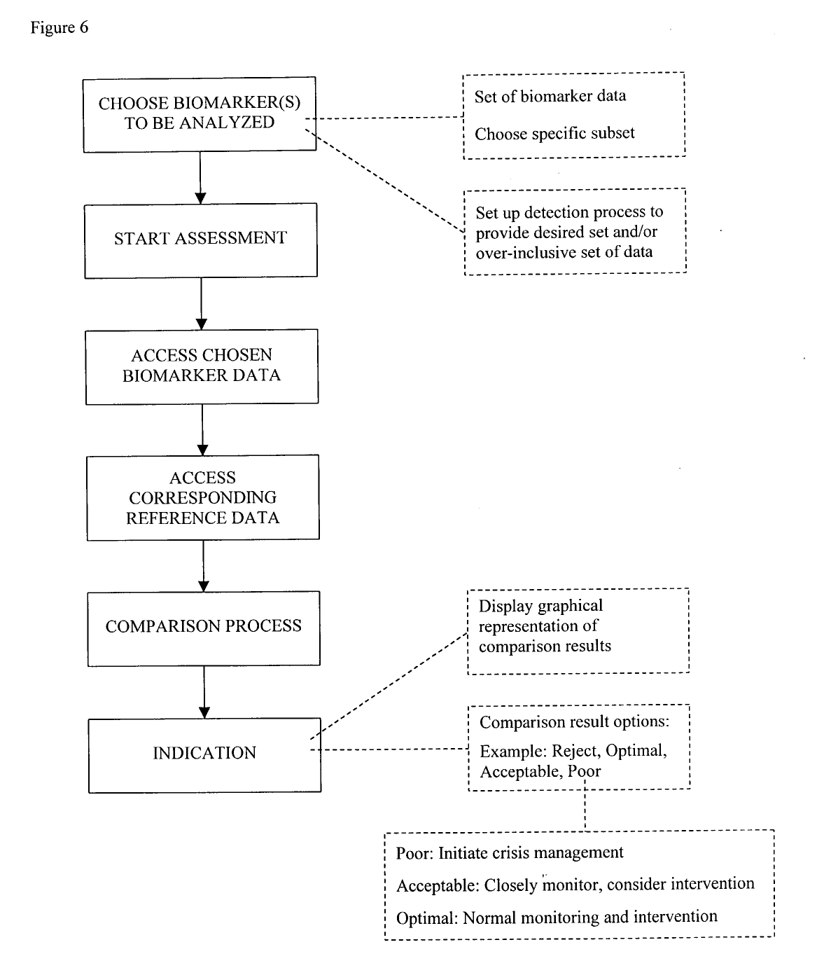

FIG. 7 shows a schematic of a computer display screen shot including a graphic representation of buttons to specify biomarker(s) to be assessed, start assessment and set comparison process options. Clicking on the “specify biomarkers” button brings up a menu permitting selection of data set and file source for the selected biomarker(s). Clicking on the “start assessment” button begins process shown in FIG. 6, which includes the comparison of the biomarker data to be assessed and reference biomarker data. Clicking on the “comparison process options” button brings up a menu for selection of options (see FIG. 8 and description below).

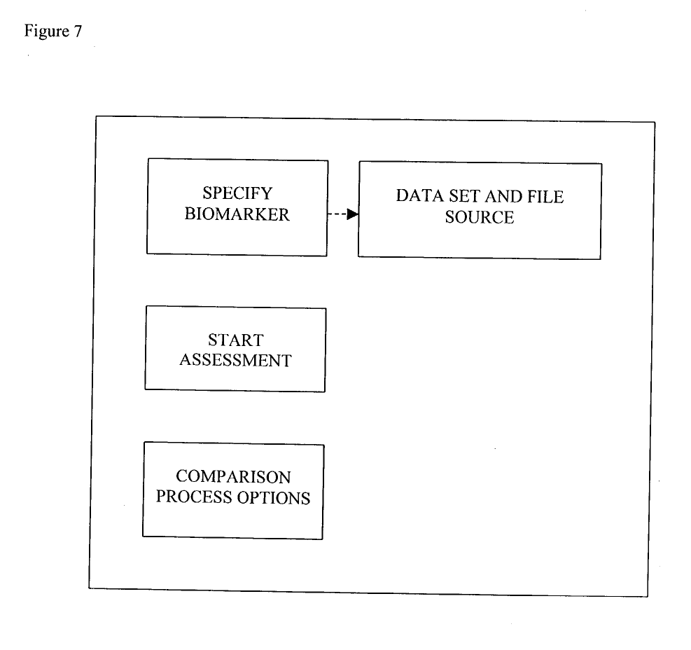

FIG. 8 shows a schematic of a computer display screen shot displaying comparison process options. Clicking on the “polypeptide biomarkers” button brings up a menu permitting a choice of biomarkers, with a further choice (check boxes) for each as to whether one wants to compare “Presence/Absence” or “Amount” of the biomarker, or both. Clicking on the “Type of Sample” button brings up a menu permitting a choice of biomarker data from transplant donors, transplant recipients, or transplant cells, tissues or organs themselves.

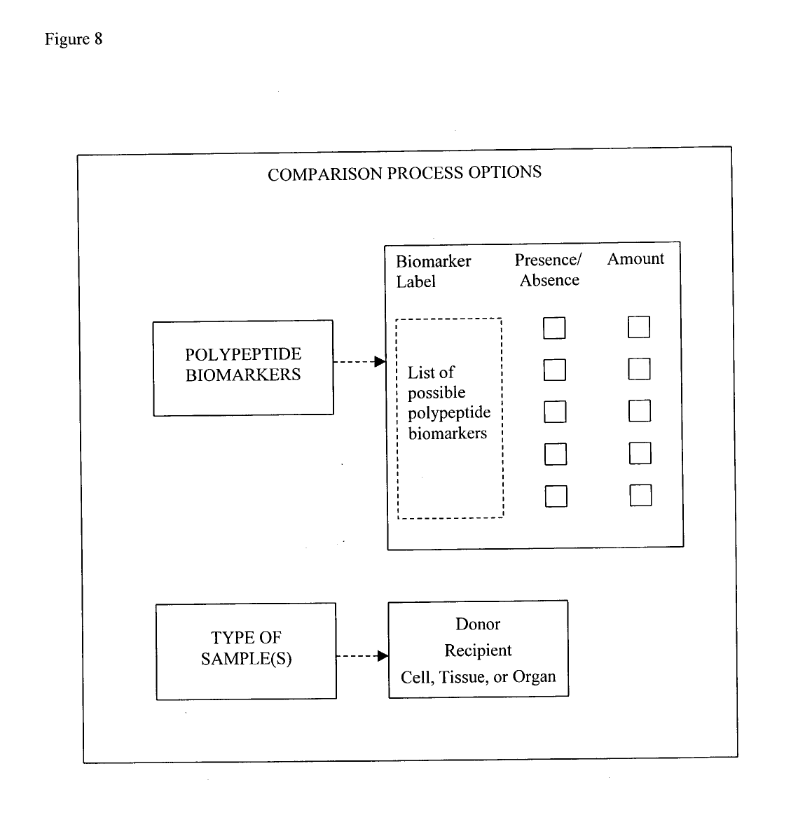

### DETAILED DESCRIPTION

The existing mechanism for determining the suitability of a tissue-matched organ for transplant relies to a great extent on imprecise analyses of the general “look and feel” of the organ, criterion highly dependent on the experience of the individuals performing the analysis. That is, in many cases the diagnostic tools utilized to assess organ quality prior to transplantation rely on a physical assessment of the tissue by the physician prior to implantation (Brasile et al., 2001, Clin. Transplant. 15: 369-374). This physical assessment typically includes evaluating organ color, rigidity, temperature, clarity of preservation solution, etc., and often results in underutilization based on nonfunctional conclusions (Pokorny et al., 1999, Transplant. Proc. 31: 2074-2076). This assessment regime serves as an unofficial standard due to limitations in availability of more quantitative diagnostic technologies. The methods and apparatus disclosed herein permit a rapid, real-time analysis of transplant status both before and after transplantation, thereby providing guidance on pre- and post-transplant decision making.

In one aspect, methods are provided for evaluating the medical condition of a cell, tissue or organ before or after it is transplanted. In this aspect, a plurality of polypeptide markers for the status of the cell, tissue of organ are detected using a protein array, preferably a protein microarray, and the presence, absence or amounts of those markers is compared with reference values. The reference represents polypeptide markers for that cell, tissue or organ from pre- and/or post transplant cells, tissues or organs for which clinical outcome, positive or negative, is known. The comparison of the markers or their pattern guides clinical decision making in the transplant process.

In another aspect, an apparatus is provided for assessing the success of the transplant of a cell, tissue or organ. In this aspect, the apparatus comprises a platform or holder to hold surface chemistry or capture agent necessary to detect a plurality of different polypeptides in a sample, a detection mechanism to determine the quantity and/or type of polypeptides bound to the platform, and a processor comprising a comparison mechanism for comparing polypeptide detection data from the sample with a reference and a mechanism for determining the condition of the cell, tissue, or organ to be transplanted based on the comparison of polypeptide detection data from the sample with the reference.

Biomarkers

An important aspect of transplant evaluation is the identification of biomarkers present in pre- or post-transplant tissues or organs that correlate with post-transplant difficulties. Thus, the identification of biomarkers that predict later problems can aid the physician in determining whether or not to go forward with a transplant, or can guide their post-operative treatment by highlighting potential problems at an early stage.

Current technology for transplant monitoring relies on indicators of complications that are sometimes not apparent for days or weeks after transplant. In one aspect, then, the methods disclosed herein measure biomarkers before and immediately after transplantation, e.g., within minutes or hours (e.g., 1, 2, 4, 8, 12, 24, 36 or 48 hours) after transplant. The identification of changes in one or more known or unknown biomarkers in this time frame provides a rapid indicator of changing status of the transplant and permits the physician to intervene much sooner than is permitted with the current methods of transplant evaluation. Thus, rapid real-time monitoring of patient and transplant status will allow for the modification of post-operative therapeutic regimes, thereby reducing or eliminating the complications associated with many transplantation procedures.

Methods are disclosed herein for the identification and use of biomarkers that indicate the status of a transplant. A “biomarker,” as the term is used herein, is a polypeptide that is an indicator for the status of a cell, tissue or organ transplant. The presence, absence or amount of the biomarker polypeptide in the transplant or in a body fluid of a donor or recipient correlates with an aspect of the health or function of the transplant. A biomarker can be a known or unknown polypeptide, as described more fully below. As used herein, a protein sample is “from a cell, tissue, or organ” if it is taken directly from the cell, tissue or organ, or if it is obtained from a body fluid (e.g., serum or urine) of an individual comprising that cell, tissue or organ or if it is taken from fluid in which the cell, tissue or organ was or is stored prior to transplant.

In one aspect, a biomarker is a known polypeptide that indicates the status of a transplant. For example, the presence and amount of a known polypeptide that becomes detectable in urine, serum or other fluid only when a transplant is under stress indicates that the cell, tissue or organ is stressed.

Examples of known biomarkers that alone or together indicate the status of tissue or organs for transplant are described below. One or more of these biomarkers can be monitored relative to their presence, absence or amount in samples from healthy, non-transplanted individuals to evaluate the status of a given transplant before or after implantation.

Kidney: Because of its function, urine is a particularly appropriate fluid to measure the status of a transplant kidney. In healthy individuals, the protein content of urine is very low, so detection of increased proteinuria is itself indicative of stress to the organ. However, biomarkers that correlate with the status of the tissue include, for example, albumin, IgA, IgG, urokinase, thyroxine binding globulin, transferrin, anti-thrombin-3, protein S, protein C, amylase, chlecalcitol, Bence Jones protein, ribonuclease and hemoglobin.

Liver: The serum levels of the following polypeptides provide examples of biomarkers for the status of liver tissue before or after transplant: aspartate aminotransferase, alanine aminotransferase, bilirubin, glutamate dehydrogenase, malate dehydrogenase, ketose-1-phosphate aldolase and lactate dehydrogenase.

Heart: The serum levels of the following polypeptides provide examples of biomarkers for the status of cardiac tissue before or after transplant: creatine kinase, aspartate aminotransferase, lactic acid dehydrogenase and fructose aldolase.

Pancreas and pancreatic islet cells: The serum levels of the following polypeptides provide examples of biomarkers for the status of pancreatic islets or tissue before or after transplant: amylase, lipase, aspartame aminotransferase, alanine aminotransferase, lactic acid dehydrogenase, alkaline phosphatase, leucine aminopeptidase, insulin, proinsulin, and glucose phosphate isomerase.

The known biomarkers can be detected, for example, following their capture with specific antibodies immobilized on an array surface. Numerous antibodies are commercially available. Alternatively, one skilled in the art can generate a monoclonal or polyclonal antibody preparation suitable for capture of a known polypeptide. Alternatively, the molecular mass of the known biomarkers is known, permitting their detection in a sample by mass spectrometry.

Alternatively, the identity of the polypeptide need not be known for it to be useful as a biomarker. In this aspect, a sample from a transplant donor, recipient, or from the tissue itself (e.g., from hypothermic storage fluid) is evaluated for the presence and/or amount of an unknown protein that correlates with the status of the transplant. To establish the ability to use unknown proteins as biomarkers, one can perform detection of proteins bound to a surface chemistry agent that binds a number of proteins, for example, an anion exchange agent. The proteins bound are then detected, for example by SELDI-TOF mass spectrometry, which generates a series of peaks corresponding to the molecular masses and amounts of the various proteins in the sample. The series of peaks provides a profile for that sample. The profiles of a number of samples from healthy donors and from transplant recipients in various stages of successful and unsuccessful transplant are then compared to identify peaks and patterns of peaks that correlate with the status of the transplant. Thus, the peaks and the proteins they represent, even though unknown, provide biomarkers for the status of the transplant. Of course, when an unknown biomarker is found to correlate closely with the status of a transplant, efforts can be focused on determining the identity of the biomarker protein, such that it can be further studied or even used as a known biomarker. Proteolytic peptide analysis and mass spectrometry can be used to identify the protein, as can microsequencing technology.

For all aspects described herein, it is assumed that a donor cell, tissue or organ to be used as a transplant has been tissue matched with the recipient. This standard process of evaluating the immunological compatibility of the donor and recipient is very well known in the art.

Samples

Any biological fluid can be monitored for biomarkers, but as noted above, samples to monitor the status of a transplant will frequently be derived from urine or blood serum or plasma of the donor or recipient. Other sample sources include, for example, saliva, the fluid in which an organ or tissue for transplant is stored prior to transplant, or small biopsies of the tissue itself. When tissue biopsies are used, they can be homogenized, for example in PBS or, alternatively, in a detergent-containing buffer to solubilize the polypeptides to be detected.

Apparatus:

In one aspect, an apparatus for assessing the success of a transplant includes an array platform to hold surface chemistry or capture agent necessary to bind a plurality of different polypeptides from a sample, a detection mechanism to determine the quantity and/or type of polypeptides bound to the platform, a processor comprising a comparison mechanism for comparing polypeptide detection data from the sample with a reference and a mechanism for determining the condition of the transplant tissue based on the comparison of polypeptide detection data from the sample with the reference.

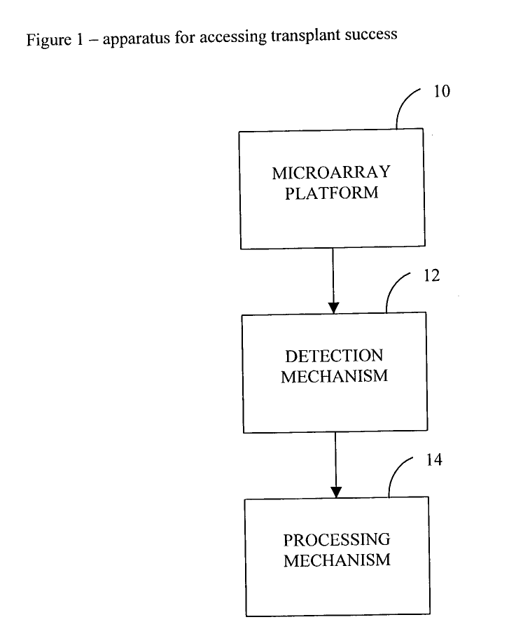

As exemplified in an embodiment shown in FIG. 1, protein microarray technology is used to detect proteins in a sample and monitor their expression levels in the sample. A microarray platform 10 uses a capture array of antibodies to detect the target proteins in the sample.

A detection mechanism 12 is used to determine the quantity and/or type of the target polypeptides in the sample that are bound to the platform. The detection mechanism can be one of a number of options described herein below.

A processing mechanism 14 processes the data gathered by detection mechanism 12 to assess the success of a transplant of a cell, tissue, or organ. Processing mechanism 14 compares the data from the sample with a reference, and determines the condition of the cell, tissue, or organ to be transplanted based on the comparison of polypeptide detection data from the sample with the reference. Based on the presence, absence or relative amount of biomarker polypeptides, a treatment determination can be made before and after the transplant of the cell, tissue, or organ.

Surface Chemistry:

The role of a given surface chemistry agent or capture agent is to bind one or more proteins present in a sample from a transplant donor or recipient or from the cell, tissue or organ itself. Once bound, the proteins can be detected to generate a profile or spectrum of the proteins present and to facilitate comparison of the profile, which in turn permits assessment of the status of the transplant.

The platform surface can be comprised of any of a number of different materials, including, for example, glass, ceramic, silicon wafer, metals, organic polymers, and beads (porous or non-porous) of cross-linked polymers (e.g., dextran, agarose, etc.) or metal. A glass, silicon or metal surface is preferred. A surface can be coated with a material, for example, gold, titanium oxide, silicon oxide, etc. that allows derivatization of the surface.

When the surface is a bead, the bead can be marked with one or more different fluorescent dyes, each dye corresponding to a particular capture agent. A sample is then exposed to a mixture of these coded beads, permitting simultaneous measurement of different proteins in a single sample volume. Detection in this aspect can be by flow cytometry. A further alternative is the use of “barcoded” nanoparticles, as described by Walt et al., 2000, Science 287: 451-454; Battersby et al., 2000, J. Am. Chem. Soc. 122: 2138-2139; Bouchez et al., 1998, Science 281: 2013-2016; and Han et al., 2001, Nature Biotechnol. 19: 631-635. These nonoparticles have “stripes” of different metals that vary in number and width, permitting a broad range of different detectable combinations of particles, each derivatized with one or more different capture agents. Detection of proteins bound to nanoparticles can be performed using, for example, mass spectrometry or fluorescence.

Where necessary the surface for the array can be derivatized with a bifunctional linker that binds a capture agent to the surface. A bifunctional linker generally has a functional group that can covalently bind with a functional group on the surface and a functional group that binds or can be activated to bind a capture agent. Examples of bifunctional linkers inculde aminoethyl disulfide and aminopropyl triethoxysilane. Alternatively, capture agents can be bound to the surface non-covalently through hydrophobic, van der Waals or ionic interactions.

A number of capture agents that bind proteins are known in the art. These include, for example, antibodies, which can be bound to a surface by any of a number of means that are well known in the art. The term “antibodies” as used herein encompasses any reactive fragment or fragments of antibodies such as Fab molecules, Fab proteins, single chain polypeptides, or the multi-functional antibodies having binding affinity for an antigen. The term includes chimeric antibodies, altered antibodies, univalent antibodies, bi-specific antibodies, monoclonal antibodies, and polyclonal antibodies.

An array can include separate spots of individual antibodies specific for known target proteins. If desired, separate spots can alternatively include more than one antibody, such that a spot can bind two or more known proteins. A variety of different antibodies are commercially available, and those of ordinary skill in the art can raise additional antibodies through standard methods. Spots of antibodies or any other capture agent can be arranged on the surface in a linear array, or, for example, in a grid arrangement that can be accessed by a detection device. Generally, any arrangement of spots that is compatible with a given detection device can be used. Arrays will comprise at least two spots comprising capture agent(s), and preferably more, e.g., 5, 10, 20, 50, 100, 250, 500 spots or more.

Additional capture agents include, for example, ion exchange and reversed-phase affinity surfaces that interact with moieties on the protein targets. A number of different surface chemistry capture agents are available in an array format on chips from Ciphergen (Fremont, Calif.). For example, carboxylate chemistry provides a negatively charged weak cation exchanger in the CM10 and WCX2 chips, and the SAX2 chip uses quaternary amine functionality for strong anion exchange. Ciphergen also sells chips with immobilized metal affinity capture agent (IMAC3), an agent that mimics reversed-phase chromatography with C16 functionality (H4), and an agent that binds through reversed-phase or hydrophobic interactions (H50), among others. Each of these agents will bind different proteins in a sample with varying degrees of selectivity. In one aspect, a single chip can have a plurality of spots with different capture agents, such that a different subset of proteins in a sample will bind to each different capture agent.

When a protein-containing sample, e.g., urine or serum, is contacted with a surface bearing a capture agent that binds proteins in that sample, proteins bind the capture agent and unbound proteins can be removed by washing. The removal of unbound proteins and other substances reduces the complexity of the sample and the resulting protein profile.

Detection Mechanisms:

In one aspect, the detection mechanism involves Surface Enhanced Laser Desorption/Ionization coupled with Time of Flight mass spectrometry, or SEDLI-TOF. SELDI is described in U.S. Pat. Nos. 5,719,060, 6,020,208, 6,027,942 and 6,124,137 which are incorporated herein by reference. The basic principle of SELDI-TOF is that a protein bound to a surface is bombarded with laser energy which induces its desorption from the surface and ionization. The time of flight of the ionized protein to a detector is recorded and converted to protein molecular weight (larger polypeptides generally have longer flight times). The amount and molecular weight of numerous proteins present in a sample can be detected simultaneously to generate a profile or spectrum of the proteins in the sample. With TOF-mass spectrometry, one can obtain information on hundreds or thousands of different proteins or peptides at a single site on an array. The method is capable of detecting nanomole to sub-femtomole quantities of protein on a spot, corresponding to millimolar to picomolar concentrations in a biological sample. Comparison of the profiles from different samples will permit the identification of protein differences between the samples, and the differences permit the assessment of the status of a transplant.

A SELDI-TOF device, the ProteinChip Reader™, is commercially available from Ciphergen (Fremont, Calif.). That device can be used essentially according to the manufacturer's instructions to generate protein profiles for samples from a transplant donor, recipient or tissue. However, exemplary conditions are as follows: The instrument can be operated in the positive ion mode with a source and detector voltage of 20 and 1.8 kV, respectively. Time-lag focusing can be used, e.g., with a pulse voltage ond lag time of 3000 V and 673 ns, respectively. Laser intensity is set at 150 (approximately 100 μJ) using a nitrogen laser emitting at 337 nm. The digitizer operates at 250 mHz. The laser traverses 66% of the target area in a linear sweep to generate each spectrum (von Eggeling et al., 2000, BioTechniques 29: 1066-1070).

The apparatus disclosed herein also includes a processor comprising a comparison mechanism for comparing polypeptide detection data from a sample with a reference. Software for comparison of spectra are available in the art. For example, Ciphergen (Fremont, Calif.) sells a software package, ProteinChip™ Software 3.0, designed for use with its ProteinChip Reader™ that performs comparisons of the mass spectra and will identify peaks that differ between samples. Analysis software and protein array chips are also available from LumiCyte (Fremont, Calif.). Software designed for interpretation and comparison of mass spectrometry data is also available from, for example, ChemSW, Inc. (N. Fairfield, Calif.), Scientific Instrument Services (Ringoes, N.J.), Agilent Technologies (Palo Alto, Calif.), BioBridge Computing (Malmo, Sweden), and Bioinformatics Solutions (Waterloo, Ontario).

Alternatives to mass spectrometric detection include fluorescent detection. WO 0004382, incorporated herein by reference, describes an ELISA-based strategy in which antibodies are arrayed on a chip and binding of protein antigen is detected by fluorescence, phosphorescence or luminescence. Labeled secondary antibodies can be employed in this or other aspects of the detection method.

Another alternative for the detection of bound proteins is surface plasmon resonance, which detects binding events by using changes in the refractive index of a surface caused by increases in mass. This approach is particularly appropriate when specific capture agents, e.g., antibodies, are used.

Additional detection alternatives include resonance light scattering (equipment and methods provided by Genicon Sciences) and atomic force microscopy (BioForce Laboratories).

Profiles/Protein Difference Maps

The pattern of the presence and/or amount of a plurality of polypeptide biomarkers in a given sample forms a biomarker profile for that sample. A comparison of the profiles from samples taken at various times before and after transplantation and in successful and ultimately unsuccessful transplants permits the creation of a protein difference map for a given cell, tissue or organ. Thus, a protein difference map is generated by identifying a biomarker pattern for a cell, tissue or organ, and comparing it to the biomarker pattern for a cell, tissue or organ at a different stage of transplantation (e.g., differing times pre-transplant, differing times post-transplant, or from an individual undergoing different degrees or stages of transplant failure or rejection). The protein difference map takes note of those proteins that appear or disappear or that increase or decrease in abundance in healthy versus ultimately unhealthy transplants. The protein difference map can also take note of trends in the amount of individual biomarkers, rather than absolute amounts of the biomarkers, that correlate with the outcome of the transplant.

Data Analysis and Decision Making Based on Profiles:

Data obtained from a protein array can be analyzed manually if needed, but are preferably analyzed by computer. Generally, any detection method for a protein array as described herein will generate a readout that can be stored and analyzed in digital form. For example, computer data acquisition from fluorescence detectors and from mass spectrometry devices is well known in the art.

As noted above, software for comparison and analysis of protein detection data are available in the art. For example, Ciphergen (Fremont, Calif.) sells a software package, ProteinChip™ Software 3.0, designed for use with its ProteinChip Reader™ that performs comparisons of the mass spectra and will identify peaks that differ between samples. Software designed for interpretation and comparison of mass spectrometry data is also available from, for example, ChemSW, Inc. (N. Fairfield, Calif.), Scientific Instrument Services (Ringoes, N.J.), Agilent Technologies (Palo Alto, Calif.), BioBridge Computing (Malmo, Sweden), and Bioinformatics Solutions (Waterloo, Ontario). Similar software products are also available for the analysis of readouts from fluorescence detectors or other detection devices.

As used herein, “a difference between the pattern observed for a transplant and a reference pattern” encompasses both similarities and differences between biomarker patterns. Thus, when there is no difference or very little difference between a reference pattern and a test sample pattern, the “difference” is indicative that the transplant outcome for the test sample will be similar to the outcome for the reference sample(s). Alternatively, where there is a wide “difference” (e.g., 50% or more higher or lower than the reference) the outcome of the test sample transplant will likely differ from the outcome of the reference pattern sample(s).

When a transplant donor or recipient sample shows a level or trend of one or more biomarkers that correlates with a level on a difference map that in turn correlates with a present or potential future problem with the transplant, treatment decisions can be guided by that information. Thus, a mechanism that determines the condition of a cell, tissue or organ before or after transplant involves a comparison of the biomarker profile from that cell, tissue or organ with a reference profile or database of profiles. Thus, a level of one or more biomarkers for a pre-transplant tissue or organ that correlates with a poor post-transplant prognosis could guide a decision not to transplant that organ.

Alternatively, a level or pattern of one or more biomarkers for a post-transplant tissue or organ that correlates with a poor post-transplant prognosis can guide a decision to aggressively treat with drugs that would otherwise not be preferred. Post-transplant monitoring of biomarkers as described herein will also permit the detection of changes in biomarkers within the recipient that herald future problems with the transplant. Because the procedure is relatively non-invasive (preferably using urine or blood testing) and because the detection is rapid (particularly when SELDI-TOF is used), the methods described herein are well suited to ongoing post-operative monitoring of transplanted tissue. As noted, software for comparison of biomarker profiles obtained by SELDI-TOF is available from Ciphergen. Software packages suitable for the analysis of profile data obtained in other ways is known to those skilled in the art and will frequently be included with a detection device.

### EXAMPLES

### Example 1

### Analysis of Biomarkers in Renal Transplant

Renal Preservation Solutions Collection

Following standard porcine nephrectomy, kidneys were gently flushed through the renal artery with HypoThermosol-FRS™ (HTS-FRS) hypothermic storage solution (BioLife Solutions, Inc. Binghamton, N.Y.) at 4° C. Following flushing, kidneys were perfused with and submerged in HTS-FRS and statically stored at 4° C. for 6 days, which is well beyond the current acceptable preservation interval of 2-3 days. During preservation, kidneys were flushed with fresh HTS-FRS every 24 hours and the effluent solution was collected during the flush procedure and stored at −80° C. for analysis.

Urinary Analysis from Transplant Recipients

Urine samples were collected from human donor and recipient patients following renal transplant at 24, 48, and 72 hours post transplant following standard biologic fluid collection NYPIRB protocol. Following collection, cells secreted into the urine were collected by centrifugation and frozen at −80° C. Upon thawing, cells were lysed in RIPA buffer (20 mM Tris (pH 8.0), 137 mM NaCl, 10% glycerol, 1% Nonidet P-40, 0.1% SDS, 0.5% deoxycholate, 2 mM EDTA) supplemented with protease inhibitors (5 mM benzamidine, 1 mM PMSF, 20 uM Pepstatin A, 7.5 mM EDTA). Cell lysate was centrifuged at 14,000 rpm for 10 minutes at 4° C., and the supernatant (cytosolic protein) was separated and stored at −20° C.

SELDI-TOF Protein Analysis

Protein Standards

Insulin and Glucagon standards were obtained from Santa Cruz Biotechnology (Santa Cruz, Calif.). Indicated amounts of protein standard were analyzed using an NP1 chip array following standard manufacturer instructions.

Sample Protein Analysis

Preservation solution analysis was performed on the HTS collected during cold storage of porcine kidney utilizing a Ciphergen Weak Cationic Exchange chip array (WCX2). The WCX2 chip bioprocessor technique was utilized to enhance protein capture from a diluted sample. Ten microliters per HTS sample was used on each chip array spot. Analysis of urine samples from transplant patients was performed on cellular protein extracts (1 μg/spot) using Ciphergen Normal Phase chip arrays (NP1). Preparation and analysis of the chips was performed following the manufacturer's standard protocol. Briefly, samples were applied to their respective chip surface spots and allowed to bind. Subsequent to the binding interval, excess unbound protein was washed off the chip with binding buffer and allowed to air dry. Following drying, Energy Absorbing Molecule (EAM) was added to each sample spot and allowed to dry again. Protein samples were then analyzed using the Ciphergen ProteinChip Reader in which sample proteins were desorbed by laser activation and time-of-flight (TOF) was recorded and converted into protein molecular weight. Protein spectra are resultant from 10-20 ProteinChip scans from each sample spot.

Data Analysis

Protein profiles from samples obtained from the SELDI-TOF ProteinChip were individually analyzed for peak identification and intensity using the Ciphergen Peaks software (version 2.0). Intensity data from corresponding individual peaks from multiple samples were combined to determine average peak intensity (±SEM). Data on protein profiles from preservation flush solutions was collected from samples obtained from three separately preserved porcine kidneys from three separate individual animals. Urine sample were provided gratis by Columbia University and the data reported represents average protein profiles and intensities (±SEM) from three individuals. Analysis of statistical significance was performed using single-factor ANOVA and P-values are reported in the text.

Results

Characterization of SELDI Protein Chip™

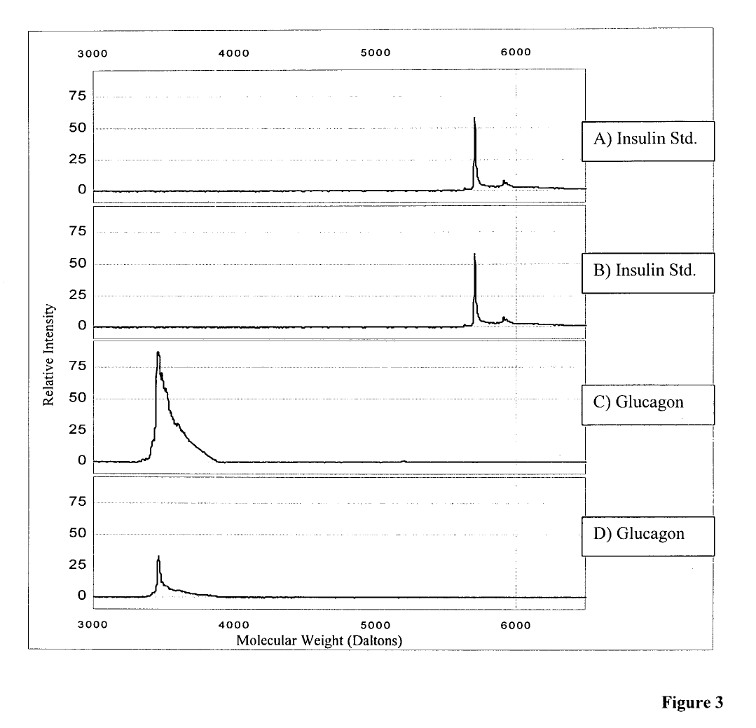

SELDI ProteinChip™ calibration and standardization was performed using purified protein standards. Purified Insulin and Glucagon samples were analyzed with the system to determine their molecular masses and compared with their reported predicted molecular masses (FIG. 3). Analysis of the Insulin standard yielded a distinctive peak at 5752 D, which closely resembled the reported molecular mass (5807 D) (FIG. 3, Spectra A and B). Similar analysis was performed using a Glucagon standard to assess calibration at multiple molecular masses and yielded a molecular mass of 3460 D, which again resembled that of the predicted mass (3482 D) (Spectra C). In addition to molecular mass determination, insulin standard analysis on duplicate chip spots revealed reproducible spectra (P<0.005) (Spectra A and B). Variation of glucagon standard concentration revealed both spectra reproducibility and sensitivity (Spectra C and D). These data revealed that the established protocol enabled reproducible molecular mass determination within 0.7% of predictive values as well as sensitivity for protein concentration comparison between samples.

Analysis of Preservation Medium

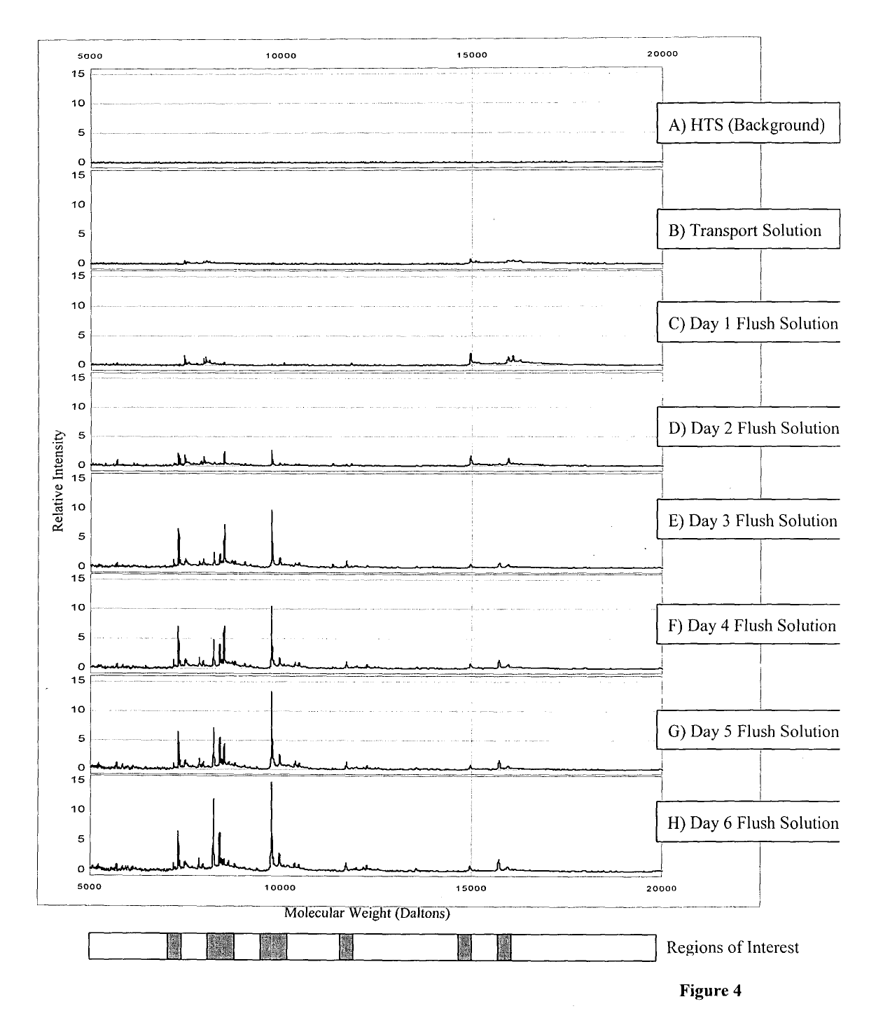

Porcine kidneys were perfused with HypoThermosol™ and statically stored at 4° C. for a period of 6 days. Kidneys were gently flushed daily with fresh HTS and the flush solution was collected for ProteinChip™ analysis (FIG. 4). Analysis of the flush solutions revealed distinct phenomic fingerprints (protein profiles) in the samples characterized by the appearance of an increasing number of unique peaks as well as an increasing intensity of existing peaks. Evaluation of the background level of HTS yielded no discernable peaks (Spectra A). Transport solution analysis [HTS surrounding the kidneys during transport (Day 1)] revealed few minor protein peaks not statistically above background (Spectra B). In comparison, analysis of the day 1 flush solution resulted in the appearance of several protein peaks ranging in molecular mass from 7350 D to 15950 daltons (D), with distinct peaks appearing around 7405, 7861, 14952, 15950 D (Spectra C). Day 2 flush solutions revealed the appearance of 3 new protein peaks at 7317, 8525, and 9758 D yielding 7 distinct peaks total (Spectra D). At 3 days of storage, the appearance of additional peaks in the flush solution continued, most notably at 8254, 9966, and 11706 D (Spectra E). Following 4-6 days of storage, no new discernable peaks were noted from those at three days, but there was a significant intensification of the existing peaks each subsequent day of analysis (Spectra F-H). In particular, the intensity of the peak at 9966 D increased from 10 (Day 3) to 13 (Day 5) to 15 (Day 6) (P<0.01) and the peak at 8254 D increased from 2 to 7 to 13 over the same interval (P<0.005) on average. Despite the overall trend toward peak intensification, it was observed that the peak at 8525 D increased from 3 to 7 between day 2 and 3 (P=0.0053) and subsequently decreased to around 5 (P=0.009) at day 5 and was at background levels by day 6 (P=0.12 from background).

Urine Protein Analysis from Transplant Patient

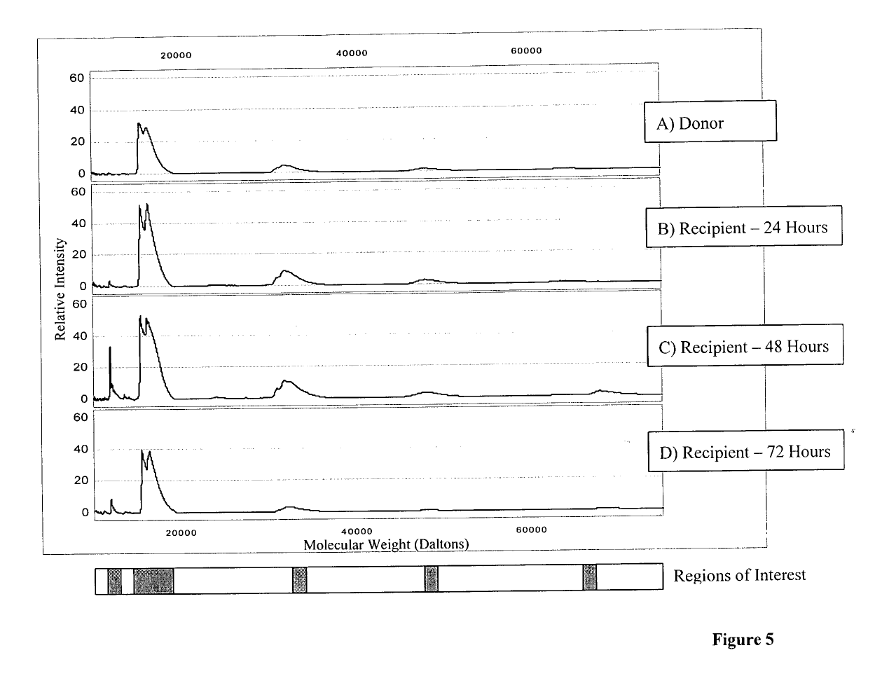

Urine from patients following kidney transplantation was collected daily over a postoperative period of 3 days and analyzed for the presence, concentration, and profile of proteins, and compared to urine protein profiles from the donors (FIG. 5). Profiling of donor urine showed the presence or several proteins, which was represented by the appearance of 4 peaks during SELDI-analysis with molecular masses of 15620, 16394, 47955, and 64005 D with intensities of 31, 28, 2 and 5, respectively (Spectra A). The 64005 D protein was present in both a 1 H⁺ and 2 H⁺ form resulting in an additional peak at an apparent molecular mass of 32560 D. Analysis of recipient urine 24 hours following transplantation revealed intensification in proteins concentration above that observed in the donor urine (Spectra B). Twenty-four hour sample analyses revealed peak intensities of 50, 54, 5, and 10 for the peaks with molecular masses of 15620, 16394, 47955, and 64005 D, respectively. The observed changes represent significant increases in protein concentration when compared to their respective peaks from the donor sample (P<0.0064). In addition to the increase, there was also the appearance of an additional peak at 11997 D with an intensity of approximately 2. Continued analysis of recipient urine at 48 hours post-transplant revealed a continued trend of increasing intensity in the 11997 D and 64005 D proteins form the 24 hour sample from 2 to 32 (P<0.001) and 10 to 12 (P=0.008), respectively (Spectra C). Peaks at 15620 D and 16394 D appeared to maintain a relatively consistent intensity over the 24 to 48 hour interval with average intensities ranging between 50-54 (P>0.27). As with the 24-hour sample, there was the appearance of a unique peak at 67919 D with an average intensity of 3 in the 48-hour post-transplant samples. Urine samples collected 72 hours post-op from recipients showed a decrease in peak intensity for all identified proteins (Spectra D). On average, all protein peaks returned to that of donor levels by 72 hours post-op (P>0.039) with the exception of the peak at 11997 D which decreased significantly from 48 hour samples from 32 to 8 (P<0.001), while remaining above that of donor levels (P=0.004).

### CONCLUSIONS

Analyses of phenomic fingerprints present in preservation solutions prior to transplantation, and in patient urine samples following transplantation, were performed. These studies show that organ degradation during hypothermic storage can be assessed and monitored through analysis of proteins released from the tissue during the preservation interval. Specifically, during storage, cellular degradation results in the release of proteins into the preservation medium, and the level and profile of these proteins can serve as an indicator for organ quality. These data also demonstrate protein profiling of urine samples from transplant recipients as a means for implant and patient monitoring.

Through the utililization of SELDI-ProteinChip microarray technology, high-throughput protein analysis allowed for the identification of unique expression profiles from individual preservation solution samples. Analysis of flush solutions from kidneys stored at 4° C. for 6 days and collected at 24 hour intervals revealed an increase in the amount and diversity of proteins released during preservation. While not wishing to be bound to a single mechanism, it is believed that the appearance and increase in the concentration of proteins in the preservation solution is a result of tissue degradation, and contains biomarkers, which serve as indicators of organ status. In particular, the significant increase in protein concentration (peak intensity) and appearance of a number of additional proteins, as discovered in the 3 day preservation solution samples in this study, represent a significant diagnostic indicator of organ transplant quality. When one considers the present generally accepted 24 to 48 hour preservation interval for kidneys (7,9), this alteration in the phenomic fingerprint may represent a significant early indicator. The analysis of preservation solution phenomic fingerprints, when correlated with transplant procedural and post-operative data, can serve as pre-operative tissue diagnostic and procedural success predictive indicator.

As with analysis of the phenomic fingerprints present in preservation solutions, similar analysis of urine from donor and recipient patients was performed to demonstrate the use of SELDI-TOF microarray technology as a diagnostic system for the evaluation of implant and recipient status. Time-course collection and analysis of urine samples revealed distinct postoperative protein profiles. In the case of the patients utilized in this study, alterations were seen at 24 and 48-hours post-transplant and by 72-hours, the profiles returned to that of or near donor profiles. When compared with available post-operative data on procedural success, it was found that all transplants were deemed successful through current evaluation techniques with few or no complications reported. The analysis of urine phenomic fingerprints can serve as a key diagnostic tool for real-time patient monitoring and can serve as a diagnostic tool for the determination of early post-operative therapeutic regimes to reduce complications currently associated with organ transplantation procedures.

The need for the development of rapid, high-throughput, real-time analytical tools and procedures will prove critical to the continued evolution of the surgical field of transplantation. The use of SELDI-TOF microarray technology for the analysis of pre-implantation organ quality as well as patient and implant post-operative status is demonstrated herein. Based upon these findings, it is shown that the application of SELDI-TOF microarray technology allows for 1) the rapid and accurate determination of phenomic fingerprints from complex biological samples, 2) phenomic fingerprints can serve as quantitative diagnostic indicators of organ quality during and following preservation, 3) analysis of urine for protein profiles represents a significant source of information regarding patient post-operative status, and 4) utilization of phenomic profiling and microarrays may facilitate the identification of specific biomarkers to serve as real-time predictive indicators for transplantation efficacy.

### REFERENCES

1) Brasile, L., Stubenitsky, B., Booster, M., et al. The Cadaveric Kidney and the Organ Shortage A perspective view. Clinical Transplantation: 15; 369-374. 2001.

2) Briggs, JD. Causes of Death After Renal Transplantation. Nephrology and Dialysis Transplantation; 16: 1545-1549. 2001. 3) Kootstra, G., Daemen, J., Oomen, A. Catagories of Non-Heart Beating Donors. Transplantation Proceedings: 70; 1254. 1995.

4) Hampel, D J., Sansome, C., Sha, M., et al. Toward Proteomics in Uroscopy: Urinary protein profiles after radiocontrast medium administration. Journal of American Society of Nephrology; 12(5): 1026-1035. 2001.

5) Hauet T, Goujon J M, Vandewalle A. To what extent can limiting cold ischaemia/reperfusion injury prevent delayed graft function? Nephrology and Dialysis Transplantation; 10:1982-1985. 2001

6) Li, X., Mohan, S., Gu, W., et al. Differential Protein Profiling in the Ear-Punched Tissue of Regeneration and Non-Regeneration Strains of Mice: A novel approach to explore the canadite genes for soft tissue regeneration. Biochimica et Biophysica Acta; 1524: 102-109. 2000.

7) Muhlbacher, F., Langer, F., Mittermayer, C. Preservation Solutions for Transplantation. Transplantation Proceedings: 31: 2069-2070. 1999.

8) Pascual, M., Theruvath, T., Kawai, T., et al. Stragaties to Improve Long-Term Outcomes After Renal Transplantation. New England Journal of Medicine: 346(8); 580-591. 2002.

9) Pokorny, H., Gruberger, K., Rockenshaub, CG., et al. Preservation of the Liver: Is it possible to extend the time of storage? Transplantation Proceedings; 31: 2074-2076. 1999.

10) St. Peter, S., Imber, C., Friend, P. Liver and Kidney Preservation by Perfusion. The Lancet: 359; 604-613. 2002.

11) Tonge, R., Shaw, J., Middleton, B., et al. Validation and Development of Fluorescence Two-Dimentional Differential Gel-Electrophoresis Proteomics Technology. Proteomics; 1: 377-396. 2001.

12) United Network of Organ Sharing (UNOS). Critical Data: U.S. facts about transplantation. www.unos.org/newsroom/critdata_main.htm.

13) Vanrenterghem, Y F. Tailoring Immunosuppressive Therapy for Renal Transplant Recipients. Pediatric Transplantation; 5: 467-472. 2001.

14) Vlahou A, Schellhammer P F, Mendrinos S, et al. Development of a novel proteomic approach for the detection of transitional cell carcinoma of the bladder in urine. American Journal of Pathology; 158(4):1491-502. 2001.

15) Von Eggeling, F., Davies, H., Lomas, L., et al. Tissue-Specific Microdissection Coupled with ProteinChip Array Technologies: Applications in cancer research. BioTechniques; 29: 1066-1070. 2000.

1. An apparatus for assessing success of a transplant of a cell, tissue, or organ, the apparatus comprising:
a holder to hold at least one of a surface chemistry and a capture agent necessary to detect a plurality of different polypeptides of a sample;
a detection mechanism to determine polypeptide detection data comprising at least one of quantity and type of polypeptides bound to the holder; and
a processor comprising a comparison mechanism to compare the polypeptide detection data from the sample with a reference and a mechanism to determine a condition of the cell, tissue, or organ to be transplanted based on the comparison of the polypeptide detection data from the sample with the reference.

2. The apparatus of claim 1, wherein the holder comprises one of a planar surface, a bead or a cylinder.

3. The apparatus of claim 1 wherein the holder comprises a microarray.

4. The apparatus of claim 1, wherein the surface chemistry or capture agent comprises an antibody.

5. The apparatus of claim 1 wherein the surface chemistry comprises an ion exchange or reversed-phase affinity agent.

6. The apparatus of claim 1, wherein the sample comprises a sample from a cell, tissue, or organ to be transplanted.

7. The apparatus of claim 1, wherein the detection mechanism comprises SELDI-TOF.

8. The apparatus of claim 1, wherein the detection mechanism comprises a labeled antibody.

9. The apparatus of claim 1, wherein the detection mechanism comprises surface plasmon resonance.

10. An apparatus for assessing success of a transplant of a cell, tissue, or organ, the apparatus comprising:
a holder to hold at least one of a surface chemistry and a capture agent necessary to detect a plurality of different polypeptides of a sample;
a detection mechanism to determine polypeptide detection data comprising at least one of quantity and type of polypeptides bound to the holder; and
a processor comprising a comparison mechanism to compare the polypeptide detection data from the sample with a reference, a mechanism to determine a condition of the cell, tissue, or organ that has been transplanted and a mechanism for making a treatment determination.

11. The apparatus of claim 10, wherein the holder comprises one of a planar surface, a bead or a cylinder.

12. The apparatus of claim 10, wherein the sample comprises a sample from the cell, tissue, or organ that has been transplanted.

13. The apparatus of claim 10, wherein the sample comprises a fluid sample from the patient who has received the cell, tissue, or organ.

14. A method of evaluating the medical condition of a cell, tissue, or organ to be used as a transplant, comprising:
providing a tissue matched cell, tissue or organ to be transplanted;
using a polypeptide array to measure the amount of a plurality of polypeptides in a sample from the cell, tissue or organ, thereby determining a pattern; and
comparing the pattern of the plurality of polypeptides from the cell, tissue, or organ to the values for a reference pattern of the plurality of polypeptides;
wherein a difference between the pattern observed for said transplant and the reference pattern is indicative of the medical condition of the transplant.

15. A method of evaluating the medical condition of a cell, tissue, or organ to be used as a transplant, comprising:
providing a cell, tissue, or organ to be used for transplant;
performing matching to assess transplant donor to recipient;
comparing a plurality of biomarkers from a cell, tissue, or organ to be used in a transplant to the values for a reference pattern of the plurality of biomarkers;
wherein a difference between the pattern observed for said transplant and the reference pattern is indicative of the medical condition of the transplant.

16. The method according to claim 15, wherein the comparing step comprises measurement of the presence, absence, or amount of the plurality of biomarkers.

17. The method according to claim 15, wherein the plurality of biomarkers is at least four.

18. The method according to claim 16, wherein the measurement is performed using a protein array.

19. The method of claim 18 wherein the protein array is a microarray.

20. The method according to claim 19, wherein the microarray comprises a plurality of antibodies.

21. The method of claim 18 wherein the array comprises an ion exchange or reversed-phase affinity agent.

22. The method according to claim 15, wherein when the cell, tissue, or organ is a kidney, the plurality of biomarkers comprises one or more of albumin, IgA, IgGm urokinase, thyroxine binding globulin, transferrin, anti-thrombin 3, protein S, protein C, amylase, chlecalcitol, Bence Jones protein, ribonuclease, and hemoglobin.

23. The method according to claim 15, wherein when the cell, tissue, or organ is a liver, the plurality of biomarkers comprises one or more of aspartate aminotransferase, alanine aminotransferase, bilirubin, glutamate dehydrogenase, malate dehydrogenase, ketose-1-phosphate aldolase, and lactate dehydrogenase.

24. The method according to claim 15, wherein when the cell, tissue, or organ is a heart, the plurality of biomarkers comprises one or more of creatine kinase, aspartate amino transferase, lactic acid dehydrogenase, and fructose aldolase.

25. The method according to claim 15, wherein when the cell, tissue, or organ is a pancreas or pancreatic islet cell, the plurality of biomarkers comprises one or more of amylase, lipase, aspartame aminotransferase, alanine aminotransferase, lactic acid dehydrogenase, alkaline phosphatase, leucine amidopeptidase, insulin, proinsulin, and glucose phosphate isomerase.

26. A method of generating a protein difference map, comprising:
identifying a first biomarker pattern from a first cell, tissue, or organ;
identifying a second biomarker pattern from a second cell tissue or organ, wherein the first and second cell, tissue or organ are at different stages of transplantation; and
comparing said first and second biomarker patterns, thereby generating a protein difference map.

27. The method according to claim 26, wherein the steps of identifying a biomarker pattern each comprise measuring the presence, absence, or amount of a plurality of biomarkers in a sample.

28. The method according to claim 26, wherein the first and second biomarker patterns comprise information regarding at least four biomarkers.

29. The method according to claim 27, wherein the biomarker pattern is identified using a micro array.

30. The method according to claim 26, wherein the first and second cell, tissue, or organ are each the same type of cell, tissue, or organ.

31. The method according to claim 30, wherein the first biomarker pattern is derived from a healthy transplant and the second biomarker pattern is derived from a rejected transplant.

32. The method according to claim 26, wherein when the cell, tissue, or organ is a kidney, the biomarker pattern comprises information regarding one or more of albumin, IgA, IgGm urokinase, thyroxine binding globulin, transferrin, anti-thrombin 3, protein S, protein C, amylase, chlecalcitol, Bence Jones protein, ribonuclease, and hemoglobin.

33. The method according to claim 26, wherein when the cell, tissue, or organ is a liver, the biomarker pattern comprises information regarding one or more of aspartate aminotransferase, alanine aminotransferase, bilirubin, glutamate dehydrogenase, malate dehydrogenase, ketose-1-phosphate aldolase, and lactate dehydrogenase.

34. The method according to claim 26, wherein when the cell, tissue, or organ is a heart, the biomarker pattern comprises information regarding one or more of creatine kinase, aspartate amino transferase, lactic acid dehydrogenase, and fructose aldolase.

35. The method according to claim 26, wherein when the cell, tissue, or organ is a pancreas or pancreatic islet cell, the biomarker pattern comprises information regarding one or more of amylase, lipase, aspartame aminotransferase, alanine aminotransferase, lactic acid dehydrogenase, alkaline phosphatase, leucine amidopeptidase, insulin, proinsulin, and glucose phosphate isomerase.

36. A method of predicting the suitability of a cell, tissue, or organ for transplant, the method comprising:
measuring the presence, absence, or amount of a plurality of polypeptide biomarkers in a cell, tissue, or organ being evaluated for transplant, to generate a biomarker pattern; and
comparing said biomarker pattern to a protein difference map representing the differences in presence, absence, or amount of said plurality of biomarkers exhibited in healthy versus unhealthy cells, tissues, or organs of the same kind, wherein said comparing predicts the suitability of said cell, tissue, or organ.

37. The method according to claim 36, wherein the measuring is performed using a microarray.

38. The method according to claim 37, wherein the microarray comprises a plurality of antibodies.

39. The method according to claim 36, wherein the plurality of biomarkers is at least four.

40. The method according to claim 36, wherein when the cell, tissue, or organ is a kidney, the plurality of biomarkers comprises one or more of albumin, IgA, IgGm urokinase, thyroxine binding globulin, transferrin, anti-thrombin 3, protein S, protein C, amylase, chlecalcitol, Bence Jones protein, ribonuclease, and hemoglobin.

41. The method according to claim 36, wherein when the cell, tissue, or organ is a liver, the plurality of biomarkers comprises one or more of aspartate aminotransferase, alanine aminotransferase, bilirubin, glutamate dehydrogenase, malate dehydrogenase, ketose-1-phosphate aldolase, and lactate dehydrogenase.

42. The method according to claim 36, wherein when the cell, tissue, or organ is a heart, the plurality of biomarkers comprises one or more of creatine kinase, aspartate amino transferase, lactic acid dehydrogenase, and fructose aldolase.

43. The method according to claim 36, wherein when the cell, tissue, or organ is a pancreas or pancreatic islet cell, the plurality of biomarkers comprises one or more of amylase, lipase, aspartame aminotransferase, alanine aminotransferase, lactic acid dehydrogenase, alkaline phosphatase, leucine amidopeptidase, insulin, proinsulin, and glucose phosphate isomerase.

44. A protein difference map made according to claim 26.

45. A computerized system to identify a condition of a cell, tissue or organ to be transplanted, the system comprising:
a stored representation of biomarker data to be assessed;
a stored representation of reference biomarker data;
a user interface;
the user interface comprising a biomarker information input mechanism to allow a user to specify information regarding the biomarker data to be assessed;
the user interface further comprising a comparison process option input mechanism to allow a user to specify a set of comparison process options;
a mechanism to compare the biomarker data to be assessed with the reference biomarker data in accordance with the specified comparison process options; and
a mechanism to indicate a likelihood of a successful transplant of a cell, tissue or organ to be transplanted based on the comparison of the biomarker data to be assessed and the reference biomarker data.

46. The computerized system of claim 45 wherein said options comprise designation of a specific subset of the biomarker data to be assessed, and designation of the source of the sample from which the reference data were obtained.

47. The computerized system of claim 46 wherein the source is one of a transplant recipient, a transplant donor, a cell to be transplanted, a tissue to be transplanted, an organ to be transplanted, a transplanted cell, a transplanted tissue, and a transplanted organ.

48. The computerized system of claim 47 wherein the source of the sample is urine, serum, plasma or saliva from a transplant donor or transplant recipient, or storage fluid for a cell, tissue or organ to be transplanted.

49. The computerized system of claim 45 wherein the mechanism to indicate a likelihood of a successful transplant displays a graphical representation of comparison results on a computer screen.

50. The computerized system of claim 45 wherein the mechanism to indicate a likelihood of a successful transplant further provides a suggested transplant approach, the approach comprising a suggestion to proceed with the transplant, a suggestion to proceed with the transplant with heightened monitoring, or a suggestion not to proceed with the transplant.

51. A computerized system to identify a condition of a transplanted cell, tissue or organ, the system comprising:
a stored representation of biomarker data to be assessed;
a stored representation of reference biomarker data;
a user interface;
the user interface comprising a biomarker information input mechanism to allow a user to specify information regarding the biomarker data to be assessed;
the user interface further comprising a comparison process option input mechanism to allow a user to specify a set of comparison process options;
a mechanism to compare the biomarker data to be assessed with the reference biomarker data in accordance with the specified comparison process options; and
a mechanism to indicate a condition of a transplanted cell, tissue or organ based on the comparison of the biomarker data to be assessed and the reference biomarker data.

52. The computerized system of claim 51 wherein said options comprise designation of a specific subset of the biomarker data to be assessed, and designation of the source of the sample from which the reference data were obtained.

53. The computerized system of claim 52 wherein the source is one of a transplant recipient, a transplant donor, a cell to be transplanted, a tissue to be transplanted, an organ to be transplanted, a transplanted cell, a transplanted tissue, and a transplanted organ.

54. The computerized system of claim 53 wherein the source of the sample is urine, serum, plasma or saliva from a transplant donor or transplant recipient, or storage fluid for a cell, tissue or organ.

55. The computerized system of claim 51 wherein the mechanism to indicate a condition displays a graphical representation of comparison results on a computer screen.

56. The computerized system of claim 51 wherein the mechanism to indicate a condition further provides a suggested treatment approach, the approach comprising a suggestion to proceed with standard monitoring, a suggestion to consider initiation of aggressive drug intervention, or a suggestion to initiate aggressive drug intervention.
